(19) United States
(12) Patent Application Publication
(10) Pub. No.: US 2016/0017400 A1
(43) Pub. Date: Jan. 21, 2016

(54) Method for improved detection of microorganisms and the like

(71) Applicants: GEOFFREY N. ROTH, GOSHEN, IN (US); JONATHAN N ROTH, GOSHEN, IN (US)

(72) Inventors: GEOFFREY N. ROTH, GOSHEN, IN (US); JONATHAN N ROTH, GOSHEN, IN (US)

(21) Appl. No.: 14/120,967

(22) Filed: Jul. 15, 2014

(51) Int. Cl. C12Q 1/04

(52) U.S. Cl. CPC C12Q 1/04; G01N 2333/195

(57) ABSTRACT

A method is provided of applying a test sample to a fibrous pad containing growth media which uses unaccelerated dispersion and deposition of the microorganism.

### FIELD OF THE INVENTION

The present invention relates to methods of detecting microbes, bacteria and the like, and, more particularly, to methods of detecting enzyme reactions which are indicative of the presence of bacteria and other microorganisms.

### BACKGROUND OF THE INVENTION

Previously, it has been suggested that membrane filtration methods can be used to detect the presence of microorganisms, such as bacteria, quantify the number of such microorganisms in a given volume of test sample, and even differentiate different types of microorganisms in the test sample. Such methods have included applying growth media to a pad that can be used to provide some of the growth media when needed. A sample test fluid may be forced through a micro-pore or nano-pore membrane using a vacuum or pressure, causing retention of microbes, and then that membrane is placed on the pad. The membrane is monitored to detect activity caused by the interaction of the growth media with the particular microorganism for which detection is sought. Typically, the activity detected would depend on the enzyme reactions associated with the maintenance or growth of the microorganism.

However, some prior methods have been found to cause damage to the bacteria in the test sample, that damage being sufficient to inhibit growth or detection of the bacteria. This can cause either longer periods of time to be used for accurate testing, and/or inaccurate test results, and/or require the use of larger test samples. Further, the prior methods can be more expensive (they usually require the use of a vacuum source, and filter funnels, etc.) and require too large a container or test module (thus, preventing a larger number of test samples from being incubated within the same incubator space).

Accordingly, it is an object of the present invention to provide an improved method of detecting microorganisms and the like. Other objects include the provision of such a detection method which is less expensive to use, more accurate, produces faster results, and produces a greater volume of results within a given time period.

### SUMMARY OF THE INVENTION

These and other objects of the present invention can be obtained by a preferred embodiment of a method of applying a test sample onto a locating surface (e.g.—micropore membrane) without having to use the traditional vacuum pump or air pressure. The membrane had been put on a pad (e.g.—cellulose) containing growth medium, but not necessarily to saturation. It is thought that the test sample disperses over the membrane and fluid from the test sample wicks through the membrane by hydraulic action to retain the microorganisms on the membrane which is monitored for microbe detection., The locator surface and/or pad may be contacted with water and/or growth medium before, during or after applying a test sample to the locating surface. In another embodiment, a relatively small amount of the test sample can be placed onto the lower surface of a standard container and an upside down membrane under a pad with media is then placed over the top of the test sample.

Other objects, advantages, and novel features of the present invention will become readily apparent to those of skill in the art from the following detailed description and claims.

### DESCRIPTION OF PREFERRED EMBODIMENTS

The present invention in particularly applicable to current testing requirements in the food and beverage industry with respect to detection of bacteria, various pathogens, and other microorganisms and the like. This invention facilitates testing as to the presence or absence of the items to be detected as well as quantitative testing as to the volume of such items and/or differentiation of the various types of items to be detected. This invention is also typically well suited to use in educational and scientific analysis or detection systems for such items.

In general, standard detection systems involve placing a container, such as a transparent 47 mm diameter dish, having therein a sample of the material to be tested into a visual detection device which allows identification and/or counting (either by the operator manually or by automated equipment) of the microorganisms, as a result of some visual differentiation between the area where the microorganisms are located on the dish and the background color of a surface in the dish where the sample is located. The visual differentiation can, for example, be as a result of an enzyme activity which is uniquely associated with the particular type of microbe which is sought to be detected. Microbes present in the test sample may produce a visual effect after interacting with nutrients. Growth may be supported or accelerated by incubation of the test sample, alone or together with a number of other test samples. The standard detections systems may also apply various inhibiting agents (e.g.—surfactants, antibiotics) or growth agents to the test sample, depending upon the purposes of the particular test.

An embodiment of the present invention presents an improvement over such standard detection systems. An embodiment of the present invention can be used to allow for faster and more accurate detection of microorganisms and the like by reducing the destruction of the microorganisms prior to their attempted detection. As used herein, “growth medium” may refer to a substance which can be used to create a detectable indicia of the presence of the item sought to be detected. In the case of microbes, for example, the growth medium can be nutrients needed by the particular type of microbes for their maintenance and/or growth, where those nutrients can be used to produce a visually distinctive indicator, at least in part as a result of enzyme reaction indicating that type of microbe. Such nutrients are well known in the industry, a wide variety of them being commercially available depending upon the particular microbe which is sought to be detected. In preferred embodiments of the present invention, the growth media is a water based, or aqueous, solution which may be applied to a pad on which a locating surface is placed, and/or to a locating surface.

As used herein, “locating surface” may refer to a structure through which water passage may occur, and which may retain items sought o be detected. Ideally, the locating surface does not allow the items to slide around to move to any significant degree, even when the dish or container is being moved, as between an incubator and a visual detection device. Ideally also, the items are dispersed over the locating surface such that accurate detection is possible. For example, if a test sample has a high microbial population, insufficiently dispersed test samples would allow one bacteria to mask the location of another bacteria, thus producing an inaccurate “count” of the bacterial presence. Some locating surfaces are well known in the industry, a wide variety of them being commercially available depending upon the particular item which is sought to be detected. As an aspect of a preferred embodiment of the present invention, the locator surface can be an item from a group comprising various pad types and various membrane types which are capable of contacting an aqueous solution and can support microorganisms and the like which are sought to be detected. For example, such surfaces can be made of various materials (e.g.—cellulose, nylon).

The inventors hereto have discovered that certain prior methods of detecting microorganisms and the like which have used membrane filtration or pour plate methods are insufficiently accurate, or more costly, or too slow to fully satisfy the needs and desires of the industry. For example, certain prior membrane filtration systems accomplish retention of microbes in the test sample onto the locating surface by placing the test sample onto a micro or nano porous membrane and then applying an acceleration force, such as differential air pressure or vacuum, to force the test sample through the membrane. It is then placed on a pad, which was previously contacted with the appropriate growth medium.

Unfortunately, some such prior methods have been found to result in the destruction of the microorganisms, such as certain microbes, which is thought to be a result of the acceleration force. Once destroyed, those microbes cannot use the growth medium, and may give negative presence results or a low bacteria count to the detector. In some cases, prior applications of these methods may have sought to compensate for such effects and/or to improve reliability by increasing the volume of the test sample and/or the incubation time. From the stand point of industrial applications, however, those increases are undesirable and can be unduly costly. Alternatively, other prior methods which are “gentler” to the bacteria may fail to provide accurate results.

One embodiment of the invention is improvement on these methods. It is thought that contact forces and the like which are found using smaller test samples and surfaces may themselves be sufficient to provide appropriate dispersion and deposition of the microorganisms onto the growth medium and that traditional acceleration forces on the test sample are to be avoided. For example, a preferred method of the present invention includes selecting a pad conventionally according to the particular item(s) to be detected, and then placing the pad into the detection container, a 47 mm diameter transparent dish, for example, and then adding to the pad an aqueous solution of the growth medium (the particular growth medium also being selected conventionally according to the particular item(s) to be detected. A conventional micro or nano porous membrane is then placed onto the top of the pad. Next, the test sample, preferably in fluid form, is placed onto the top of the membrane. The amount of test sample should not be such that it flows over the edge of the membrane, nor should it completely cover the top surface of the membrane. In some instances the amount of the test sample can be one milliliter or less.

The test sample may be allowed to disperse laterally over the top surface of the membrane of its own accord. Fluid from the test sample is thought to wick through the membrane onto the pad. Thereafter, the disk is incubated and/or monitored for detection of the desired items.

In another embodiment of the present invention, the test sample is first placed onto the lower interior surface of the detection container, again preferably in fluid form and in a volume which does not cover the bottom surface of the detection container. Next, an upside down micropore membrane under a pad (previously contacted with the growth medium) is placed onto the test sample. That placement itself can allow for appropriate dispersion of the test sample laterally over the lower interior surface of the detection container. It is thought that the nature of the membrane and the pad and the fluidic and/or evaporative forces associated with the interaction of the test sample and the growth medium can allow for movement of nutrients to the test items at least sufficient to provide the normal enzyme reactions of bacteria, for example, which thereafter would permit application of conventional detection and counting techniques, through the bottom of the detection container.

Various adaptations of the present invention are anticipated for given applications. For example, depending upon the particular item to be detected, a “gum” surface can be used to provide the growth medium and to locate the items to be detected thereon. Also, two embodiments can be combined, such that the barrier “locating surface,” (e.g.—membrane) is used on one side of a pad for one test sample, and the other side of the pad is contacted to a second barrier (e.g.—membrane) or both sides of a pad are a barrier locating surface for a second test sample. In each case, it is thought that hydraulic wicking can sufficiently support both appropriate dispersion and deposition of microbes onto each barrier (e.g.—membrane) surface. Also, the method of the present invention is not limited to the use of dishes as the detection container. In certain applications, films, for example, may be sufficient as the detection container.

Accordingly, the present invention has been described herein with respect to certain preferred embodiments, but the spirit and scope of this invention are limited only by the scope of the following claims:

What is claimed is:

1. A method of preparing test samples for item detection, comprising the steps of:
applying a test sample to a surface which is capable of positively locating the item to be detected without use of a supplemental force, such as a vacuum, such that the test sample contacts nutrients.

2. The method according to claim 1 wherein said surface is a porous membrane and is on a pad with nutrients, and then test sample is applied to the top of that membrane, and a portion of the test sample passes through that membrane to contact said pad.

3. The method according to claim 2 wherein membrane is thereafter monitored for indications of the presence of items to be detected by virtue of some indicia appearing on the membrane.

4. The method according to claim 3 wherein the following preliminary steps are used:
a second test sample is placed on the inside surface of a detection container, the volume of that second test sample being insufficient to cover said upper surface,
then, an upside down membrane is placed on top of that second test sample, and
then, the steps set forth in the sequence indicated in claims 1, 3 and 4 are applied.
